(19) United States
(12) Patent Application Publication
(10) Pub. No.: US 2009/0118321 A1
(43) Pub. Date: May 7, 2009

(54) 5-Urea Substituted Naphthalimide Derivatives, Methods of Production and Pharmaceutical Compositions for Treating Cancer

(75) Inventors: Eric Van Quaquebeke, Woluwe St Lambert (BE); Gentiane Simon, Bruxelles (BE); Mohamed El Yazidi, Ganshoren (BE); Jerome Tuti, Jemeppe-sur-Sambre (BE); Laurent Van den Hove, Forest (BE); Francis Darro, Waterloo (BE); Robert Kiss, St-Pieters Leeuw (BE)

Correspondence Address: Davidson, Davidson & Kappel, LLC, 485 7th Avenue, 14th Floor, New York, NY, 10018, US

(73) Assignee: Unibioscreen S.A., Brussels (BE)

(21) Appl. No.: 12/227,090

(22) PCT Filed: May 7, 2007
(86) PCT No.: PCT/EP2007/003991

§ 371 Date: Nov. 5, 2008

Related U.S. Application Data
(60) Provisional application No. 60/746,560, filed on May 5, 2006.

(30) Foreign Application Priority Data
May 5, 2006 (GB) 0608900.7

(51) Int. Cl. A61K 31/473; C07D 221/06; A61P 35/00

(52) U.S. Cl. 514/296; 546/100

(57) ABSTRACT

Novel ureyl-substituted naphthalimide derivatives, pharmaceutically acceptable salts thereof and solvates thereof, are useful for making pharmaceutical compositions for the treatment of cell proliferative diseases such as cancer. The invention also provides methods for making such derivatives through hydrolysis of known compounds.

### TECHNICAL FIELD

The present invention relates to novel substituted naphthalimide derivatives, methods for their production and their pharmaceutical uses as anti-tumor agents, in particular in the form of pharmaceutical compositions including them as active principles in the prevention and/or treatment of various forms of cancer.

### BACKGROUND OF THE INVENTION

Various kinds of substituted naphthalimides, including amonafide, are known in the art as having anti-tumour effect or other useful biological activity. In particular WO 2005/105753 discloses naphthalimide derivatives having a specific substitution pattern which are active in the treatment of cell proliferation diseases such as cancer.

Although the level of activity found for amonafide was and continues to be of high interest, this material does have significant deficiencies which indicate the continuing need for agents with improved properties. In the first place, amonafide was found to be too toxic for some patients: in particular it has produced substantial myelotoxicity leading to some deaths in patients receiving five daily doses of the drug. In addition, it was shown that amonafide had only moderate activity in leukemia models in mice. Also, it was shown that amonafide has no activity in human tumour xenografts in mice with colon, lung and mammary cancers. Thus, while amonafide shows significant biological activity, it does not have a substantially broad spectrum of activity in murine tumour models. Ajani et al. in Invest New Drugs (1988) 6:79-83 has shown that amonafide has poor activity when tested in primary human solid tumours in vitro.

Although the clinical activity of antiproliferative agents such as amonafide against certain forms of cancers can be shown, improvement in tumor response rates, duration of response, decrease of myelotoxicity and ultimately patient survival are still sought. There is also a need in the art for improving the efficacy of antiproliferative treatments in humans by providing suitable combinations of new drugs with conventional antineoplastic agents.

In view of the above-mentioned shortcomings of amonafide and the like, there is a need in the art for naphthalimide derivatives demonstrating a more promising activity/side effects balance.

### SUMMARY OF THE INVENTION

The present invention is based on the first unexpected finding that a naphthalimide derivative being substituted with a ureyl group at position 5 of the naphthalimidyl moiety is useful in the treatment of cell proliferation disorders, can be made in an efficient manner through a limited number of reaction steps, and does not exhibit some of the drawbacks of the previously known similar derivatives. The present invention is also based on the unexpected finding that such ureyl-substituted naphthalimide derivatives are easily accessible in good yield through hydrolysis of other known substituted naphthalimide derivatives. The present invention is also based on the unexpected finding that such ureyl-substituted naphthalimide derivatives exhibit a satisfactory chemical stability and can easily be formulated into medicaments, e.g. as a suspension in the form of nanoparticles or as a solution in the form of a salt.

### DEFINITIONS

As used herein with respect to a substituting group, and unless otherwise stated, the term “alkyl” means straight and branched chain saturated acyclic hydro-carbon monovalent radicals having from 1 to 4 carbon atoms such as, for example, methyl, ethyl, propyl, n-butyl, 1-methylethyl (isopropyl), 2-methylpropyl (isobutyl), and 1,1-dimethylethyl (ter-butyl).

As used herein with respect to a member of a substituting group, and unless otherwise stated, the term “alkylene” means a divalent hydrocarbon radical corresponding to the above defined alkyl such as, but not limited to, methylene, bis(methylene), tris(methylene), tetramethylene, and the like.

As used herein with respect to a substituting group, and unless otherwise stated, the terms “alkoxy” and “alkylthio” refer to substituents wherein an alkyl group such as defined hereinabove is attached to an oxygen atom or a divalent sulfur atom through a single bond, such as but not limited to methoxy, ethoxy, propoxy, butoxy, isopropoxy, sec-butoxy, tert-butoxy, thiomethyl, thioethyl, thiopropyl, thiobutyl, and the like.

As used herein with respect to a substituting atom, and unless otherwise stated, the term “halogen” means any atom selected from the group consisting of fluorine, chlorine, bromine and iodine.

As used herein with respect to a substituting group, and unless otherwise stated, the term “haloalkyl” refers to an alkyl radical (such as above defined) in which one or more hydrogen atoms are independently replaced by one or more halogens (preferably fluorine, chlorine or bromine) such as, but not limited to, difluoromethyl, trifluoromethyl, trifluoroethyl, dichloromethyl and the like.

As used herein and unless otherwise stated, the term “solvate” includes any combination which may be formed by a ureyl-substituted naphthalimide (isoquinolinedione) derivative of this invention with a suitable inorganic solvent (e.g. hydrates formed from water) or a suitable organic solvent such as, but not limited to, alcohols, ketones, esters and the like.

As used herein and unless otherwise stated, the term “anti-migratory” refers to the ability of a pharmaceutical ingredient to stop the migration of cells away from the neoplastic tumor tissue and thus to reduce the colonization of new tissues by these cells.

The term “cell proliferative disorder” as used herein refers, but is not limited, to any type of cancer or other pathologic condition involving cell proliferation such as leukemia, lung cancer, colorectal cancer, central nervous system (CNS) cancer, melanoma, ovarian cancer, kidney cancer, prostate cancer, breast cancer, glioma, bladder cancer, bone cancer, sarcoma, head and neck cancer, liver cancer, testicular cancer, pancreatic cancer, stomach cancer, oesophageal cancer, bone marrow cancer, duodenum cancer, eye cancer (retinoblastoma) and lymphoma.

### BRIEF DESCRIPTION OF THE DRAWINGS

FIG. 1 shows compound-induced hematotoxicity on platelets by a compound of this invention, as compared to amonafide.

FIG. 2 shows P-gp ATPase activity as measured by spectrophotometry for a compound of this invention, as compared to amonafide.

FIG. 3 shows (A) drug-induced pro-autophagic effects evaluated by quantification of acidic vesicular organelles (revealed as red fluorescent staining), and (B) drug-induced lysosomal membrane permeabilization (LMP) evaluated following acridine orange staining and quantification of green fluorescent staining, at different concentrations of a compound of this invention.

FIG. 4 shows senescence-associated β-galactosidase activity in DU-145 human prostate cancer cells induced by a compound of this invention, as compared to doxorubicin.

### DETAILED DESCRIPTION OF THE INVENTION

In a first aspect, the present invention provides a group of substituted naphthalimide derivatives represented by the structural formula (I)

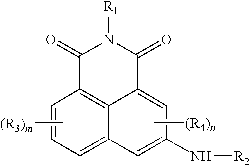

wherein:
R₁ is mono- or di-C₁₋₄ alkylamino-C₁₋₄ alkyl;
each of the substituents R₃ and R₄ is independently selected from the group consisting of hydrogen, halogen, C₁₋₄ alkyl, C₁₋₇ alkoxy, C₁₋₄ alkylthio, nitro, cyano, amino, protected amino and halo C₁₋₄ alkyl;
m is the number of substituents R₃ and ranges from 0 to 3;
n is the number of substituents R₄ and ranges from 0 to 2; and
R₂ is CONH₂;
   and/or a pharmaceutically acceptable salt thereof and/or a solvate thereof and/or a metabolite thereof.

Metabolites of the derivatives represented by the structural formula (I) include, but are not limited to, the following:

mono-N-oxides and di-N-oxides thereof;

derivatives wherein R₂ is CONHOH; and

derivatives wherein R₃ and/or R₄ is hydroxyl.

Alternatively, mono- and di-N-oxides of the naphthalimide derivatives of this invention can be directly synthesized by treating a derivative represented by the structural formula (I) with an oxidizing agent such as, but not limited to, hydrogen peroxide (e.g. in the presence of acetic acid) or a peracid such as chloroperbenzoic acid.

The above defined novel compounds have in common the structural feature that the amino group of an amino-substituted naphthalimide (isoquinolinedione) such as, but not limited to, amonafide is substituted by an ureyl group or, in a metabolised form thereof, an ureyl N-oxide group.

In a preferred embodiment of this first aspect, the present invention relates to a sub-group of compounds wherein:
n=0 (when R₄ is not hydrogen), and/or
m=0 (when R₃ is not hydrogen), and/or
m=2, both substituents R₃ being adjacent and together with the carbon atoms to which they are attached forming a phenyl group, and/or
R₁ is an alkylene radical having from 1 to 3 carbon atoms and linked to a dimethylamino or diethylamino group, and/or
R₂ is CONH₂;
   and/or a pharmaceutically acceptable salt thereof and/or a solvate thereof and/or a metabolite thereof.

In another preferred embodiment of this first aspect, the present invention relates to a sub-group of compounds wherein:
n=m=0 (when R₃ and R₄ are not hydrogen), and/or
R₁ is an alkylene radical having 1 or 2 carbon atoms and linked to a dimethylamino or diethylamino group, and/or
R₂ is CONH₂;
   and/or a pharmaceutically acceptable salt thereof and/or a solvate thereof and/or a metabolite thereof.

In yet another preferred embodiment of this first aspect, the present invention relates to a sub-group of compounds, salts, solvates or metabolites thereof, wherein R₃ is not nitro when m equals 1. In yet another preferred embodiment of this first aspect, the present invention relates to a sub-group of compounds, salts, solvates or metabolites thereof, wherein R₃ and/or R₄ is selected from the group consisting of hydrogen, halogen, C₁₋₄ alkyl, C₁₋₇ alkoxy, C₁₋₄ alkylthio, cyano, amino, acylamino and halo C₁₋₄ alkyl.

In another preferred embodiment, the present invention relates to N-{2-[2-(dimethylamino)ethyl]-1,3-dioxo-2,3-dihydro-1H-benzo[de]isoquinolin-5-yl}urea, a salt or a metabolite thereof.

In a second aspect, the present invention provides a method for the production of ureyl-substituted naphthalimide (isoquinolinedione) derivatives represented by the structural formula (I) by hydrolysing a 5-substituted amonafide or amonafide derivative wherein the 5-substituent thereof is selected in such a way that it can be converted into an ureyl group through hydrolysis. Suitable 5-substituted amonafide derivatives for hydrolysis include, but are not limited to, compounds having the structural formula (II)

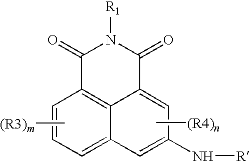

wherein:
each of m, n, R₁, R₃ and R₄ is as defined with respect to the structural formula (I), and
R′ is C₁₋₄ alkoxyamidocarbonyl or C₁ haloalkylamidocarbonyl.

Some compounds having the above structural formula (II) are already known e.g. from WO 2005/105753, but have been accessible only in very moderate yields, e.g. as a product of reacting amonafide with a C₁₋₄ alkoxycarbonyl isocyanate such as ethoxycarbonyl isocyanate or a C₁ haloalkylcarbonyl isocyanate such as trichloroacetyl isocyanate or trifluoroacetyl isocyanate. Therefore another aspect of the present invention is to design reaction conditions which permit to access these intermediates in better yields. A method for this purpose is one wherein said reaction of amonafide with a C₁₋₄ alkoxycarbonyl isocyanate or a C₁ haloalkylcarbonyl isocyanate is performed under conditions including:
the presence of a solvent, said solvent being selected from the group consisting of ethers (e.g. diethyl ether), ketones (e.g. 2-butanone or methylethylketone) and halogenated hydrocarbons (preferably having at most 2 carbon atoms and/or at least one chlorine atom, e.g. dichloromethane), and/or
a temperature below 0° C., e.g. a temperature ranging from about −30° C. to about −5° C., and/or
a molar excess of said C₁₋₄ alkoxycarbonyl isocyanate or C₁ haloalkylcarbonyl isocyanate, and/or
quenching the reaction after its completion by adding water to the reaction mixture, thus avoiding (when a molar excess of C₁₋₄ alkoxycarbonyl isocyanate or C₁ haloalkylcarbonyl isocyanate is used) the formation of undesirable cyclisation by-products.

When one or more of the above reaction conditions are used, compounds having the above structural formula (II) can be obtained in significantly better yields, within the same or a shorter reaction time, than according to the prior art. The skilled person is capable of readily determining which combination of the afore-said process features, depending upon parameters such as the exact nature of R′, R₁, R₃ and R₄, is able to provide optimal yield within the shortest possible reaction time.

Hydrolysing a 5-substituted amonafide or amonafide derivative wherein the 5-substituent thereof can be converted into an ureyl group such as, but not limited to, compounds having the structural formula (II), may be performed either under acidic conditions or under basic conditions. The skilled person readily understands that this kind of hydrolysis is susceptible, depending upon parameters such as, but not limited to, pH, temperature, the kind of acid or base being used and the kind of solvent for the reaction mixture, to produce amonafide as a by-product which then has to be separated from the desired compound having the structural formula (I). The determination of optimal conditions for minimizing the formation of amonafide is within the general knowledge of the person skilled in the art. An advantage of the present invention is that it has proved quite easy to keep the proportion of residual amonafide in the final product below 3% by weight.

In a third aspect, the present invention provides a pharmaceutical composition comprising:
a therapeutically effective amount of an ureyl-substituted naphthalimide (isoquinolinedione) derivative represented by the structural formula (I), and/or a pharmaceutically acceptable salt thereof and/or a solvate thereof and/or a metabolite thereof; and
one or more pharmaceutically acceptable carriers.

In another aspect, the present invention provides combined preparations containing at least one ureyl-substituted naphthalimide (isoquinolinedione) derivative represented by the structural formula (I) and/or a pharmaceutically acceptable salt thereof and/or a solvate thereof and/or a metabolite thereof, and one or more antineoplastic drugs, preferably in the form of synergistic combinations as detailed below.

In another aspect, the invention relates to the unexpected finding that substituted naphthalimide (isoquinolinedione) derivatives represented by the general formula (I), and/or a pharmaceutically acceptable salt thereof and/or a solvate thereof and/or a metabolite thereof, have significantly higher biological activity, especially with respect to tumour cells, than amonafide while avoiding many of the above-mentioned drawbacks of amonafide. In particular, the ureyl-substituted naphthalimide derivatives according to the present invention have a significant anti-migratory effect. Migration refers to the biological process whereby cells migrate from a neoplastic tumor tissue and colonize new tissues, using blood or lymphatic vessels as major routes of migration, this biological process being also known as the metastatic process. Based on this finding, the present invention provides a method for treating and/or preventing tumours in humans. More specifically, the invention relates to a method of treatment of a host with a cellular proliferative disease, comprising contracting said host with an effective amount of an ureyl-substituted naphthalimide (isoquinolinedione) derivative represented by the structural formula (I), and/or a pharmaceutically acceptable salt thereof and/or a solvate thereof and/or a metabolite thereof.

In another embodiment, the invention provides the use of ureyl-substituted naphthalimide (isoquinolinedione) derivatives represented by the structural formula (I), and/or a pharmaceutically acceptable salt thereof and/or a solvate thereof and/or a metabolite thereof, as anti-tumour agents.

In another particular embodiment, the invention relates to a group of ureyl-substituted naphthalimide (isoquinolinedione) derivatives, as well as pharmaceutical compositions comprising them as an active principle, having the above structural formula (I) and being in the form of a pharmaceutically acceptable salt. The latter include any therapeutically active non-toxic salt which compounds having the structural formula (I) are able to form with a salt-forming agent. Such addition salts may conveniently be obtained by treating the ureyl-substituted naphthalimide (isoquinolinedione) derivatives of the invention with an appropriate salt-forming acid or base. For instance, ureyl-substituted naphthalimide (isoquinolinedione) derivatives having basic properties may be converted into the corresponding therapeutically active, non-toxic acid salt form by treating the free base form with a suitable amount of an appropriate acid following conventional procedures. Examples of such appropriate salt-forming acids include, for instance, inorganic acids resulting in forming salts such as but not limited to hydrohalides (e.g. hydrochloride and hydrobromide), sulfate, nitrate, phosphate, diphosphate, carbonate, bicarbonate, and the like; and organic monocarboxylic or dicarboxylic acids resulting in forming salts such as, for example, acetate, propanoate, hydroxyacetate, 2-hydroxypropanoate, 2-oxopropanoate, lactate, pyruvate, oxalate, malonate, succinate, maleate, fumarate, malate, tartrate, citrate, methanesulfonate, ethanesulfonate, benzoate, 2-hydroxybenzoate, 4-amino-2-hydroxybenzoate, benzene-sulfonate, p-toluene-sulfonate, salicylate, p-aminosalicylate, pamoate, bitartrate, camphorsulfonate, edetate, 1,2-ethanedisulfontate, fumarate, glucoheptonate, gluconate, glutamate, hexylresorcinate, hydroxynaphthoate, hydroxyethanesulfonate, mandelate, methylsulfate, pantothenate, stearate, as well as salts derived from ethanedioic, propandioic, butandioic, (Z)-2-butenedioic, (E)2-butenedioic, 2-hydroxybutanedioic, 2,3-dihydroxybutane-dioic, 2-hydroxy-1,2,3-propane-tricarboxylic, cyclohexane-sulfamic acid and the like.

Ureyl-substituted naphthalimide (isoquinolinedione) derivatives having the structural formula (I) having acidic properties may be converted in a similar manner into the corresponding therapeutically active, non-toxic base salt form. Examples of appropriate salt-forming bases include, for instance, inorganic bases like metallic hydroxides such as, but not limited to, those of alkali and alkaline-earth metals like calcium, lithium, magnesium, potassium and sodium, or zinc, resulting in the corresponding metal salt; nitrogen-containing organic bases such as, but not limited to, ammonia, alkylamines, benzathine, hydrabamine, arginine, lysine, N,N′-dibenzylethylenediamine, chloroprocaine, choline, diethanolamine, ethylenediamine, N-methylglucamine, procaine and the like.

Reaction conditions for treating the ureyl-substituted naphthalimide (isoquinolinedione) derivatives (I) of this invention with an appropriate salt-forming acid or base are similar to standard conditions involving the same acid or base but different organic compounds with basic or acidic properties, respectively. Preferably, in view of its use in a pharmaceutical composition or in the manufacture of medicament for treating cell proliferative diseases, the pharmaceutically acceptable salt will be designed, i.e. the salt-forming acid or base will be selected, so as to impart greater water-solubility, lower toxicity, greater stability and/or slower dissolution rate to the ureyl-substituted naphthalimide (isoquinolinedione) derivative of this invention.

The present invention further provides the use of an ureyl-substituted naphthalimide (isoquinolinedione) derivative represented by the structural formula (I), or a pharmaceutically acceptable salt or a solvate thereof and/or a metabolite thereof, as a biologically-active ingredient, i.e. an active principle, especially as a medicine or a diagnostic agent or for the manufacture of a medicament or a diagnostic kit. In particular the said medicament may be for the prevention or treatment of a pathologic condition selected from the group consisting of cell proliferative disorders.

The compounds according to this invention are highly active against several types of cancers. Therefore, due to their favorable pharmacological properties, the compounds according to this invention are particularly suitable for use as medicaments or in the preparation of medicaments and combined preparations for the treatment of patients suffering from diseases associated with cell proliferation, more especially for treating cancer.

Any of the uses mentioned above may also be restricted to a non-medical use (e.g. in a cosmetic composition), a non-therapeutic use, a non-diagnostic use, a non-human use (e.g. in a veterinary composition), or exclusively an in-vitro use, or a use with cells remote from an animal.

The invention further relates to a pharmaceutical composition comprising:
(a) one or more ureyl-substituted naphthalimide (isoquinolinedione) derivative represented by the structural formula (I), and/or a pharmaceutically acceptable salt thereof and/or a solvate thereof and/or a metabolite thereof, and
(b) one or more pharmaceutically acceptable carriers.

In another embodiment, this invention provides combined preparations, preferably synergistic combinations, of one or more ureyl-substituted naphthalimide (isoquinolinedione) derivatives represented by the structural formula (I), and/or a pharmaceutically acceptable salt thereof and/or a solvate thereof and/or a metabolite thereof, with one or more biologically-active drugs being preferably selected from the group consisting of antineoplastic drugs. As is conventional in the art, the evaluation of a synergistic effect in a drug combination may be made by analysing the quantification of the interactions between individual drugs, using the median effect principle described by Chou et al. in Adv. Enzyme Reg. (1984) 22:27. Briefly, this principle states that interactions (synergism, additivity, antagonism) between two drugs can be quantified using the combination index (hereinafter referred as CI) defined by the following equation:

$${CI}_{x} = \frac{{ED}_{x}^{1 c}}{{ED}_{x}^{1 a}} + \frac{{ED}_{x}^{2 c}}{{ED}_{x}^{2 a}}$$

wherein EDₓ is the dose of the first or respectively second drug used alone (1a, 2a), or in combination with the second or respectively first drug (1c, 2c), which is needed to produce a given effect. The said first and second drug have synergistic or additive or antagonistic effects depending upon CI<1, CI=1, or CI>1, respectively. As will be explained in more detail herein-below, this principle may be applied to a number of desirable effects such as, but not limited to, an activity against cell proliferation.

The invention further relates to a composition or combined preparation having synergistic effects against cell proliferation and containing:
(a) one or more antineoplastic drugs, and
(b) at least one ureyl-substituted naphthalimide (isoquinolinedione) derivative represented by the structural formula (I), and/or a pharmaceutically acceptable salt thereof and/or a solvate thereof and/or a metabolite thereof, and
(c) optionally one or more pharmaceutical excipients or pharmaceutically acceptable carriers,
   for simultaneous, separate or sequential use in the treatment or prevention of cell proliferative disorders.

Suitable antineoplastic drugs for inclusion into the synergistic antiproliferative pharmaceutical compositions or combined preparations of this invention are preferably selected from the group consisting of alkaloids, alkylating agents (including but not limited to alkyl sulfonates, aziridines, ethylenimines, methylmelamines, nitrogen mustards and nitrosoureas), antibiotics, antimetabolites (including but not limited to folic acid analogs, purine analogs and pyrimidine analogs), enzymes, interferon and platinum complexes. More specific examples include acivicin; aclarubicin; acodazole; acronine; adozelesin; aldesleukin; altretamine; ambomycin; ametantrone; amino-glutethimide; amsacrine; anastrozole; anthramycin; asparaginase; asperlin; azacitidine; azetepa; azotomycin; batimastat; benzodepa; bicalutamide; bisantrene; bisnafide; bizelesin; bleomycin; brequinar; bropirimine; busulfan; cactinomycin; calusterone; caracemide; carbetimer; carboplatin; carmustine; carubicin; carzelesin; cedefingol; chlorambucil; cirolemycin; cisplatin; cladribine; crisnatol; cyclophosphamide; cytarabine; dacarbazine; dactinomycin; daunorubicin; decitabine; dexormaplatin; dezaguanine; diaziquone; docetaxel; doxorubicin; droloxifene; dromostanolone; duazomycin; edatrexate; eflomithine; elsamitrucin; enloplatin; enpromate; epipropidine; epirubicin; erbulozole; esorubicin; estramustine; etanidazole; ethiodized oil I 131; etoposide; etoprine; fadrozole; fazarabine; fenretinide; floxuridine; fludarabine; fluorouracil; flurocitabine; fosquidone; fostriecin; gemcitabine; Gold 198; hydroxyurea; idarubicin; ifosfamide; ilmofosine; interferon α-2a; interferon α-2b; interferon α-n1; interferon α-n3; interferon β-1a; interferon γ-1b; iproplatin; irinotecan; lanreotide; letrozole; leuprolide; liarozole; lometrexol; lomustine; losoxantrone; masoprocol; maytansine; mechlorethamine; megestrol; melengestrol; melphalan; menogaril; mercaptopurine; methotrexate; metoprine; meturedepa; mitindomide; mitocarcin; mitocromin; mitogillin; mitomalcin; mitomycin; mitosper; mitotane; mitoxantrone; mycophenolic acid; nocodazole; nogala-mycin; ormaplatin; oxisuran; paclitaxel; pegaspargase; peliomycin; pentamustine; peplomycin; perfosfamide; pipobroman; piposulfan; piroxantrone; plicamycin; plomestane; porfimer; porfiromycin; prednimustine; procarbazine; puromycin; pyrazofurin; riboprine; rogletimide; safingol; semustine; simtrazene; sparfosate; sparsomycin; spirogermanium; spiromustine; spiroplatin; streptonigrin; streptozocin; strontium 89 chloride; sulofenur; talisomycin; taxane; toxoid; tecogalan; tegafur; teloxantrone; temoporfin; teniposide; teroxirone; testolactone; thiamiprine; thioguanine; thiotepa; tiazofurin; tirapazamine; topotecan; toremifene; trestolone; triciribine; trimetrexate; triptorelin; tubulozole; uracil mustard; uredepa; vapreotide; verteporfin; vinblastine; vincristine; vindesine; vinepidine; vinglycinate; vinleurosine; vinorelbine; vinrosidine; vinzolidine; vorozole; zeniplatin; zinostatin; zorubicin; and their pharmaceutically acceptable salts.

Other suitable anti-neoplastic compounds for inclusion into the synergistic antiproliferative pharmaceutical compositions or combined preparations of this invention include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; anti-androgens such as, but not limited to, benorterone, cioteronel, cyproterone, delmadinone, oxendolone, topterone, zanoterone; anti-estrogens such as, but not limited to, clometherone; delmadinone; nafoxidine; nitromifene; raloxifene; tamoxifen; toremifene; trioxifene and their pharmaceutically acceptable salts; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin; azasetron; azatoxin; azatyrosine; baccatin III derivatives; balanol; batimastat; BCR/ABL antagonists; benzochlorins; benzoylstaurosporine; β-lactam derivatives; β-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-aminotriazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; clomifene and analogues thereof; clotrimazole; collismycin A and B; combretastatin and analogues thereof; conagenin; crambescidin 816; cryptophycin and derivatives thereof; curacin A; cyclopentanthraquinones; cycloplatam; cypemycin; cytarabine; cytolytic factor; cytostatin; dacliximab; dehydrodidemnin B; deslorelin; dexifosfamide; dexrazoxane; dexverapamil; didemnin B; didox; diethylnorspermine; dihydro-5-azacytidine; dihydrotaxol; dioxamycin; diphenyl spiromustine; docosanol; dolasetron; doxifluridine; droloxifene; dronabinol; duocarmycin SA; ebselen; ecomustine; edelfosine; edrecolomab; elemene; emitefur; epristeride; estrogen agonists and antagonists; exemestane; filgrastim; finasteride; flavopiridol; flezelastine; fluasterone; fluorodaunorunicin; forfenimex; formestane; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idoxifene; idramantone; ilomastat; imidazoacridones; imiquimod; immunostimulant peptides; insulin-like growth factor-1 receptor inhibitor; interferon agonists; iobenguane; iododoxorubicin; ipomeanol; irinotecan; iroplact; irsogladine; isobengazole; isohomohalicondrin B; itasetron; jasplakinolide; kahalalide F; lamellarin-N; leinamycin; lenograstim; lentinan; leptolstatin; leukemia inhibiting factor; leuprorelin; levamisole; liarozole; lissoclinamide; lobaplatin; lombricine; lonidamine; lovastatin; loxoribine; lurtotecan; lutetium texaphyrin; lysofylline; mannostatin A; marimastat; masoprocol; maspin; matrilysin inhibitors; matrix metalloproteinase inhibitors; merbarone; meterelin; methioninase; metoclopramide; MIF inhibitors; mifepristone; miltefosine; mirimostim; mitoguazone; mitolactol; mitonafide; mitotoxin fibroblast growth factor-saporin; mofarotene; molgramostim; human chorionic gonadotrophin monoclonal antibody; mopidamol; mycaperoxide B; myriaporone; N-acetyldinaline; N-substituted benzamides; nafarelin; nagrestip; naloxone; pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; octreotide; okicenone; onapristone; ondansetron; ondansetron; oracin; osaterone; oxaliplatin; oxaunomycin; palauamine; palmitoylrhizoxin; pamidronic acid; panaxytriol; panomifene; parabactin; pazelliptine; peldesine; pentosan; pentostatin; pentrozole; perflubron; perillyl alcohol; phenazinomycin; phenylacetate; phosphatase inhibitors; picibanil; pilocarpine; pirarubicin; piritrexim; placetin A and B; plasminogen activator inhibitor; propyl bis-acridone; prostaglandin J2; proteasome inhibitors; protein kinase C inhibitors; protein tyrosine phosphatase inhibitors; purine nucleoside phosphorylase inhibitors; purpurins; pyrazoloacridine; raltitrexed; ramosetron; ras farnesyl protein transferase inhibitors; ras inhibitors; ras-GAP inhibitors; retelliptine; rhenium 186 etidronate; rhizoxin; retinamide; rohitukine; romurtide; roquinimex; rubiginone B1; ruboxyl; saintopin; sarcophytol A; sargramostim; sizofuran; sobuzoxane; sodium borocaptate; sodium phenylacetate; solverol; somatomedin binding protein; sonermin; sparfosic acid; spicamycin D; splenopentin; spongistatin 1; squalamine; stem-cell division inhibitors; stipiamide; stromelysin inhibitors; sulfinosine; suradista; suramin; swainsonine; tallimustine; tamoxifen; tauromustine; tazarotene; tecogalan; tellurapyrylium; telomerase inhibitors; temozolomide; tetrachlorodecaoxide; tetrazomine; thaliblastine; thiocoraline; thrombopoietin; thymalfasin; thymopoietin receptor agonist; thymotrinan; thyroid stimulating hormone; tin ethyl etiopurpurin; titanocene; topsentin; tretinoin; triacetyluridine; tropisetron; turosteride; tyrosine kinase inhibitors; tyrphostins; ubenimex; urogenital sinus-derived growth inhibitory factor; urokinase receptor antagonists; variolin B; velaresol; veramine; verdins; verteporfin; vinxaltine; vitaxin; zanoterone; zilascorb; and their pharmaceutically acceptable salts.

Synergistic activity of the pharmaceutical compositions or combined preparations of this invention against cell proliferation may be readily determined by means of one or more tests such as, but not limited to, the measurement of the radioactivity resulting from the incorporation of ³H-thymidine in culture of tumour cell lines. For instance, different tumour cell lines may be selected in order to evaluate the anti-tumour effects of the tested compounds, such as but not limited to:
RPMI1788: human Peripheral Blood Leucocytes (PBL) Caucasian tumor line,
Jurkat: human acute T cell leukemia,
EL4: C57Bl/6 mouse lymphoma, or
THP-1: human monocyte tumour line.
   Depending on the selected tumour cell line, different culture media may be used, such as for example:
for RPMI1788 and THP-1: RPMI-1640+10% FCS+1% NEAA+1% sodium pyruvate+5×10⁻⁵ mercapto-ethanol+antibiotics (G-418 0.45 μg/ml);
for Jurkat and EL4: RPMI-1640+10% FCS+antibiotics (G-418 0.45 μg/ml).

In a specific embodiment of the synergy determination test, the tumour cell lines are harvested and a suspension of 0.27×10⁶ cells/ml in complete medium is prepared. The suspensions (150 μl) are added to a microtiter plate in triplicate. Either complete medium (controls) or the tested compounds at the test concentrations (50 μl) are added to the cell suspension in the microtiter plate. The cells are incubated at 37° C. under 5% CO₂ for about 16 hours. ³H-thymidine is added, and the cells incubated for another 8 hours. The cells are harvested and radioactivity is measured in counts per minute (CPM) in a β-counter. The ³H-thymidine cell content, and thus the measured radioactivity, is proportional to the proliferation of the cell lines. The synergistic effect is evaluated by the median effect analysis method as disclosed herein-before.

The pharmaceutical composition or combined preparation with synergistic activity against cell proliferation according to this invention may contain the ureyl-substituted naphthalimide (isoquinolinedione) derivative having the structural formula (I), and/or a pharmaceutically acceptable salt thereof and/or a solvate thereof and/or a metabolite thereof, over a broad content range depending upon the precise contemplated use and the expected effect of the preparation. Generally, the ureyl-substituted naphthalimide (isoquinolinedione) derivative content of the combined preparation is within the range of about 0.1 to about 99.9% by weight, preferably from 1 to 99% by weight, more preferably from 5 to 95% by weight.

The pharmaceutical compositions and combined preparations according to this invention may be administered orally or in any other suitable fashion. Oral administration is preferred and the preparation may have the form of a tablet, aqueous dispersion, dispersable powder or granule, emulsion, hard or soft capsule, syrup, elixir or gel. The dosing forms may be prepared using any method known in the art for manufacturing these pharmaceutical compositions and may comprise as additives sweeteners, flavoring agents, coloring agents, preservatives and the like. Carrier materials and excipients are detailed hereinbelow and may include, inter alia, calcium carbonate, sodium carbonate, lactose, calcium phosphate or sodium phosphate; granulating and disintegrating agents, binding agents and the like. The pharmaceutical composition or combined preparation of this invention may be included in a gelatin capsule mixed with any inert solid diluent or carrier material, or has the form of a soft gelatin capsule, in which the ingredient is mixed with a water or oil medium. Aqueous dispersions may comprise the biologically active composition or combined preparation in combination with a suspending agent, dispersing agent or wetting agent. Oil dispersions may comprise suspending agents such as a vegetable oil. Rectal administration is also applicable, for instance in the form of suppositories or gels. Injection (e.g. intravenously, intramuscularly or intraperitoneally) is also applicable as a mode of administration, for instance in the form of injectable solutions or dispersions, depending upon the disorder to be treated and the condition of the patient.

Unless otherwise stated, the term “pharmaceutically acceptable carrier or excipient” as used herein in relation to pharmaceutical compositions and combined preparations means any material or substance with which the active principle(s), i.e. the ureyl-substituted naphthalimide of this invention and optionally the antineoplastic drug, may be formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders.

Suitable pharmaceutical carriers for use in the said pharmaceutical compositions of the invention, and efficient ways for their formulation, are well known to those skilled in the art of pharmacology. There is no particular restriction to their selection within the present invention although, due to the usually low or very low water-solubility of the pteridine derivatives of this invention, special attention will be paid to the selection of suitable carrier combinations that can assist in properly formulating them in view of the expected time release profile. Suitable pharmaceutical carriers include additives such as wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, dissolving, spray-drying, coating and/or grinding the active ingredients, in a one-step or a multi-steps procedure, with the selected carrier material and, where appropriate, the other additives such as surface-active agents. The pharmaceutical compositions of the present invention may also be prepared by micronization, for instance in view to obtain them in the form of microspheres usually having a diameter of about 1 to 10 μm, namely for the manufacture of microcapsules for controlled or sustained release of the biologically active ingredient(s).

Suitable surface-active agents for use in the pharmaceutical compositions of the present invention are preferably non-ionic, cationic and/or anionic materials having good emulsifying, dispersing and/or wetting properties. Such suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkyl-arylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alkanolamine salts of dodecyl-benzene sulphonic acid or dibutyl-naphtalenesulphonic acid or a naphtalene-sulphonic acid/formaldehyde condensation product. Also suitable for carrying out the invention are the corresponding phosphates, e.g. salts of phosphoric acid ester and an adduct of p-nonyl-phenol with ethylene and/or propylene oxide, or phospholipids. Suitable phospholipids for this purpose include, but are not limited to, the natural (originating from animal or plant cells) or synthetic phospholipids of the cephalin or lecithin type such as e.g. phosphatidyl-ethanolamine, phosphatidylserine, phosphatidylglycerine, lysolecithin, cardiolipin, dioctanyl-phosphatidylcholine, dipalmitoylphosphatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with polypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants for carrying out this invention include, but are not limited to, quaternary ammonium salts, preferably halides, having four hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2^{nd} ed. (Hanser Verlag, Vienna, 1981) and “Encyclopaedia of Surfactants” (Chemical Publishing Co., New York, 1981).

Structure-forming, thickening or gel-forming agents may also be included into the pharmaceutical compositions and combined preparations of the invention. Suitable such agents include in particular, but are not limited to, highly dispersed silicic acid, such as the product commercially available under the trade name Aerosil; bentonites; tetraalkyl ammonium salts of montmorillonites (e.g., products commercially available under the trade name Bentone), wherein each of the said alkyl groups may contain from 1 to 20 carbon atoms; cetostearyl alcohol and modified castor oil products (e.g. the product commercially available under the trade name Antisettle).

Gelling agents which may also be included into the pharmaceutical compositions and combined preparations of the present invention include, but are not limited to, cellulose derivatives such as carboxymethylcellulose, cellulose acetate and the like; natural gums such as arabic gum, xanthum gum, tragacanth gum, guar gum and the like; gelatin; silicon dioxide; synthetic polymers such as carbomers, and mixtures thereof in any suitable proportions. Gelatin and modified celluloses represent a preferred class of gelling agents.

Other optional excipients which may also be present in the pharmaceutical compositions and combined preparations of the present invention include, but are not limited to, additives such as magnesium oxide; azo dyes; organic and inorganic pigments such as titanium dioxide; UV-absorbers; stabilisers; odor masking agents; viscosity enhancers; antioxidants such as, for example, ascorbyl palmitate; sodium bisulfite, sodium metabisulfite and the like, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid; flavoring agents such as natural vanillin; buffers such as citric acid and acetic acid; extenders or bulking agents such as silicates, diatomaceous earth, magnesium oxide or aluminum oxide; densification agents such as magnesium salts; and mixtures thereof.

Additional ingredients may be included in order to control the duration of action of the biologically-active ingredient in the compositions and combined preparations of the invention. Control release compositions may thus be achieved by selecting appropriate polymer carriers such as for example polyesters, polyamino-acids, polyvinyl-pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethyl-cellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition or combined preparation of the invention may also require protective coatings.

Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol, complexing agents such as cyclodextrins and the like, and mixtures thereof.

Since, in the case of combined preparations including the ureyl-substituted naphthalimide (isoquinolinedione) derivative having the structural formula (I), and/or a pharmaceutically acceptable salt thereof and/or a solvate thereof and/or a metabolite thereof, and an antineoplastic drug, both ingredients do not necessarily bring out their synergistic therapeutic effect directly at the same time in the patient to be treated, the said combined preparation may be in the form of a medical kit or package containing the two ingredients in separate but adjacent form. In the latter context, each ingredient may therefore be formulated in a way suitable for an administration route different from that of the other ingredient, e.g. one of them may be in the form of an oral or parenteral formulation whereas the other is in the form of an ampoule for intravenous injection or an aerosol.

The present invention further relates to a method for preventing or treating a cell proliferative disorder in a patient, preferably a mammal, more preferably a human being. The method of this invention consists of administering to the patient in need thereof an effective amount of an ureyl-substituted naphthalimide (isoquinolinedione) derivative having the structural formula (I), and/or a pharmaceutically acceptable salt thereof and/or a solvate thereof and/or a metabolite thereof, optionally together with an effective amount of an antineoplastic drug, or a pharmaceutical composition comprising the same, such as disclosed above in extensive details. The effective amount of the ureyl-substituted naphthalimide (isoquinolinedione) derivative is usually in the range of 0.01 mg to 20 mg, preferably 0.1 mg to 5 mg, per day per kg bodyweight for humans. Depending upon the pathologic condition to be treated and the patient's condition, the said effective amount may be divided into several sub-units per day or may be administered at more than one day intervals. The patient to be treated may be any warm-blooded animal, preferably a human being, suffering from said pathologic condition.

The following examples are intended to illustrate several embodiments of the present invention, including the preparation, pharmaceutical formulation and biological evaluation of the ureyl-substituted naphthalimides, without limiting its scope in any way.

### EXAMPLE 1

### Preparation of ethyl({2-[2-(dimethylamino)ethyl]-1,3-dioxo-2,3-dihydro-1H-benzo[de]isoquinolin-5-yl}amino)carbonylcarbamate

1.086 g of amonafide were dissolved in 80 mL of 2-butanone at −20° C. under nitrogen atmosphere. Then 880 mg of ethoxycarbonyl isocyanate (2 molar equivalents) dissolved in 2 mL of 2-butanone were carefully added during 5 minutes by using a dropping funnel. Reaction temperature was maintained at −20° C. during 25 minutes under stirring. The reaction mixture was then warmed up to 45° C. during 40 minutes, after which time 250 μL of water was added. After this reaction quenching step, the precipitate formed was filtrated at 40° C. on paper. After drying, 1.162 g of the expected product (structural formula below) was obtained (yield: 76%). High performance liquid chromatography (hereinafter referred as HPLC) showed a purity above 95.6%. A slight amount of amonafide (about 2%) was still present.

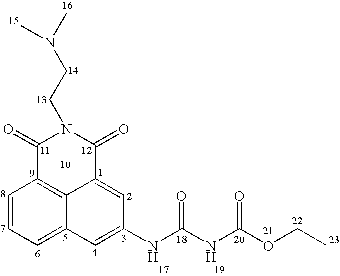

The desired product was characterised by:
proton nuclear magnetic resonance (300 MHz, CDCl₃), showing the same peaks as in example 4 of WO 2005/105753, and
electron-spray ionisation mass spectrum: showing a peak at M+H⁺=399; and the presence of an adduct at 2M+H⁺=797.

### EXAMPLE 2

### Preparation of N-{2-[2-(dimethylamino)ethyl]-1,3-dioxo-2,3-dihydro-1H-benzo[de]isoquinolin-5-yl}urea

100 mg of the compound of example 1 were dissolved in 100 mL of NaOH 0.1 M. The reaction mixture was warmed up to reflux and maintained at this temperature during 1 hour. The mixture was analysed by HPLC, showing the presence of the expected urea (structural formula below) as the major product (yield 76%).

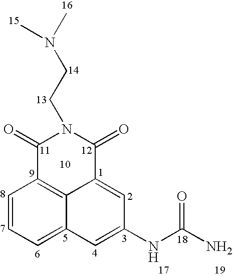

This product was characterised by the following techniques:

proton nuclear magnetic resonance (RMN ¹H, 300 MHz, DMSO) showing peaks at: 9.40 (NH-17, bs); 8.53 (H-2, d, J=1.8); 8.48 (H-4, d, J=1.8); 8.26-8.32 (H-6 and H-7, m); 6.18 (NH2-19, bs); 4.14 (H-14, t, J=6.6); 2.51 (H-13, m); and 2.21 (H-15 and H-16, s) ppm;

¹³C NMR (75.4 MHz, DMSO, TMS as internal standard) showing peaks at 37.5 (CH2, C-13); 49.9 (2×CH3, C-15 and C-16); 57.0 (CH, C14); 119.0 (CH, C-arom); 122.2 (C, C-arom); 122.9 (C, C-arom); 123.5 (C, C-arom); 123.9 (CH, C-arom); 127.8 (CH, C-arom); 128.6 (CH, C-arom); 132.7 (C, C-arom); 133.8 (CH, C-arom); 140.3 (C, C-arom); 156.5 (C, C-18); and 163.8 (C, C-12); 164.0 (C, C-11) ppm; and

electron-spray ionisation mass spectrum: showing a peak at M+H⁺=327 and an adduct at 2M+H⁺=653.

### EXAMPLE 3

### Effect on Overall Cell Growth

Tests were performed in order to rapidly, i.e. within 5 days, measure the effect of the compound of example 2 on the overall cell growth. The test measures the number of metabolically active living cells that are able to transform the yellow product 3-(4,5-dimethylthiazol-2-yl)-2,5-diphenyl tetrazolium bromide (herein referred as MTT) into the blue product formazan dye by mitochondrial reduction. The amount of formazan obtained at the end of the experiment, measured by means of a spectrophotometer, is directly proportional to the number of living cells. Optical density determination thus enables a quantitative measurement of the effect of the investigated compounds as compared to the control condition (untreated cells) and/or to other reference compounds.

Six human cancer cell lines described in table 1 were used in the following MTT tests. These cell lines cover six histological cancer types, being prostate, glioma, pancreas, colon, lung and breast cancers. Cells were allowed to grow in 96-well micro-wells with a flat bottom with an amount of 100 μl of cell suspension per well with 1,000 to 4,000 cells/well depending on the cell type used. Each cell line was seeded in a well known MEM 10% serum culture medium.

TABLE 1

| Cell line | ATCC code | tissue | literature reference |
|---|---|---|---|
| PC3 | CRL-1435 | Prostate | Invest. Urol. 17: 16-23, 1979; |
| | | | Cancer Res. 40: 524-534, 1980 |
| U-373MG | HTB-17 | Glioma | Acta Pathol. Microbial. Scand. 74: |
| | | | 465-486, 1968 |
| BxPC3 | CRL-1687 | Pancreas | Cancer Invest. 4: 15-23, 1986; |
| | | | Clin. Lap. Med. 2: 567-578, 1982 |
| LoVo | CCL-229 | Colon | Exp. Cell Res. 101: 414-416, 1976; |
| | | | J. Natl. Cancer Inst. 61: 75-83, |
| | | | 1978; Cancer Res. 39: 2630-2636, |
| | | | 1979 |
| A549 | CCL-185 | Lung | J. Natl. Cancer Inst. 51: 1417- |
| | | | 1423, 1973; Int. J. Cancer 17: |
| | | | 62-70, 1976 |
| MCF-7 | HTB-22 | Breast | J. Natl. Cancer Inst. 51: 1409- |
| | | | 1416, 1973 |

The detailed experimental procedure was as following: after a 24-hour period of incubation at 37° C., the culture medium was replaced by 100 μl of fresh medium in which the tested compound was previously dissolved, at the following molar concentrations: 10⁻⁹ M, 5.10⁻⁹ M, 10⁻⁸ M, 5.10⁻⁸ M, 10⁻⁷ M, 5.10⁻⁷ M, 10⁻⁶ M, 5.10⁻⁶ M and 10⁵ M. Each experiment was repeated 6 times.

After 72 hours of incubation at 37° C. with (experimental conditions) or without (control condition) the compound to be tested, the medium was replaced by 100 μl MTT dissolved in RPMI (1640 without phenol red) at a concentration of 1 mg/ml. The micro-wells were subsequently incubated during 3 hours at 37° C. and centrifuged at 400 g during 10 minutes. MTT was removed and formazan crystals formed were dissolved in 100 μl DMSO. The micro-wells were shaken for 5 minutes and read on a spectrophotometer at wavelengths of 570 nm (maximum formazan absorbance) and 630 nm (back-ground noise).

For each experimental condition, the mean optical density was calculated, as well as the percentage of remaining living cells in comparison with the control.

Table 2 below shows the IC₅₀ values for the compound of example 2. IC₅₀ represents the range of molar concentrations at which the tested compound inhibited by 50% the overall tumor cells growth.

TABLE 2

| Cell line | IC₅₀ (M) |
|---|---|
| PC3 | 10⁻⁵-5.10⁻⁶ |
| U-373MG | 10⁻⁵-5.10⁻⁶ |
| BxPC3 | 10⁻⁵-5.10⁻⁶ |

### EXAMPLE 4

### Effect on Cell Migration

Cells of different cancer lines, i.e. U-373 MG (Glioma cancer) and A549 (Lung cancer) were seeded on culture flask 48 hours before the migration experiment. On the test day, cells were treated with or without the compound of example 2 in closed Falcon dishes containing a buffered medium at a controlled temperature (37.0±0.1° C.) for 12 or 22 hours, as noted in the right column of table 3. The compound of example 2 was used at three non-cytotoxic concentrations (10⁶ M, 10⁻⁷M, 10⁻⁸ M). Migration of the cells was observed by means of a CCD-camera mounted on a phase-contrast microscope. Statistical analysis of the migration, with the non-parametric Mann-Whitney test, was established for 25% and 50% of the most motile cells and for the entire cell population. Table 3 below shows the anti-migratory effect of the tested compound.

TABLE 3

| Cell line | Maximum effects | Conditions |
|---|---|---|
| U-373MG | −29% | 22 hours on the 25% of |
| | p < 0.001 | the most motile cells, at |
| | | 10⁻⁷ M |
| U-373MG | −24% | 22 hours on the 50% of |
| | p < 0.001 | the most motile cells, at |
| | | 10⁻⁷ M |
| U-373MG | −20% | 22 hours on the 100% of |
| | | cells, at 10⁻⁷ M |

The data of table 3 demonstrate that the compound of example 2 induced a decrease in the migration level of U-373 MG cancer cells at the non-cytotoxic concentrations used in this study. In particular, this compound shows a statistically significant inhibition of cell migration.

### EXAMPLE 5

### Nano-Particles Suspension Formulation

A nanoparticle suspension is used for the formulation of the compound of example 2. For this approach, selected excipients (in particular tensio-active agents) including polysorbate 80 (Tween 80), Texapon K12 (SDS), PVA (Polyvinyl alcohol), Lutrol F68 (Poloxamer 188), Lutrol F127 (Poloxamer 407), Hydroxypropyl-β-cyclodextrine, Sodium taurocholate and other phospholipids (Lipoid S PC-3 and Phopholipon 90 H) are used.

After selecting the excipients and their amounts, the suspension containing the compound of example 2 is prepared by simply adding the designed quantity thereof into the desired volume of water. The suspension is then submitted to turax at 24000 rpm at low temperature for preliminary particle size reduction. The suspension is then submitted to an emulsiflex homogenisator at high pressure. Three cycles of homogenisation at different pressures may be used to obtain the expected size particle, e.g. the first cycle is performed at 7000 psi during 7 minutes, the second cycle at 12000 psi during 8 minutes and finally the last cycle at about 21000-24000 psi during 30 minutes. A determination of the particle size distribution is then made by Lazer diffraction, 5 measures being made with 20 seconds between each measure. The average of these 5 measures represents the particle size distribution of the suspension.

### EXAMPLE 6

### Preparation of 2,2,2-trichloro-N—[({2-[2-(dimethylamino)ethyl]-1,3-dioxo-2,3-dihydro-1H-benzo[de]isoquinolin-5-yl}amino)carbonyl]acetamide

A 3-necked 12 L round bottomed flask equipped with mechanical stirrer, reflux condenser, cooling ice bath, dropping funnel and temperature controller was charged under nitrogen with amonafide (90 g) and 3.6 L of methylethylketone (MEK). The resulting suspension was cooled to −10° C. and a solution of 120 g of trichloroacetylisocyanate (0.64 mole) in 600 mL MEK was added drop-wise over 35 min, while maintaining the temperature bellow −5° C. The reaction mixture was stirred between −10° C. and −5° C. for 3 h, followed by the slow addition of 2.8 mL of water. The mixture was allowed to warm at room temperature and the resulting solids isolated by filtration, washed on the filter with 100 mL of MEK, and air dried for two days to give 147 g of the desired compound (yield: 97%; purity: 98.1%). The characterizing spectra of this compound were the same as described in example 2 of WO 2005/105753.

### EXAMPLE 7

### Alternative Preparation of N-{2-[2-(dimethylamino)ethyl]-1,3-dioxo-2,3-dihydro-1H-benzo[de]isoquinolin-5-yl}urea

A 3-necked 22-L round bottomed flask equipped with mechanical stirrer, reflux condenser, and temperature controller was charged with the compound of example 6 (250 g) and 7.5 L of a 5% solution of K₂CO₃ in water. The mixture was cooled to 10° C. with (ice bath), and then 7.5 L of methanol (MeOH) was added in one portion. The temperature rose to 20° C. The flask was removed from the ice bath and stirring was continued at ambient temperature until most of the starting material dissolved (about 30-45 min). The mixture was quickly filtered (clarified), to remove the small amounts of unreacted materials and other mechanical impurities. The mixture was stirred at room temperature for 2 hours, and 4 L of MeOH was charged in one portion. The mixture was heated at 54-56° C. for 3 hours. Reaction progress was monitored by HPLC to ensure completion. The reaction mixture was cooled (ice bath) and kept at 8-10° C. for 2 hours. The obtained solid was isolated by filtration, washed on the filter (2×100 mL of water), and then dried in air for 2 days to give 156 g (yield: 90%) of the desired compound (HPLC purity: 99.47%). The characterizing spectra of this compound were the same as described in example 2 herein-above.

### EXAMPLE 8

### Lactic Acid Based Solution Formulation

A liquid solution of the compound produced according to example 2 or example 7 was obtained as follows.

First a 2% by volume Lactic Acid solution was made as follows: to a 50 mL volumetric flask, 40 mL of 0.9% NaCl solution for injection and, using a Class A—TD Pipette, 1.18 mL of lactic acid, 85% ACS reagent were added. The volume was then adjusted with 0.9% NaCl solution for injection to 50 mL and the whole was mixed by inversion.

To a 25 mL volumetric flask, 700 mg of the compound of examples 2 and 7 were weighed accurately. To this specified quantity, 10 mL of 0.9% NaCl solution for injection and 8.89 mL of the aforementioned aqueous 2% Lactic Acid solution were added. The solution obtained was stirred vigorously and sonicated for 10 minutes. The pH of the solution was between 6.4-6.6. The pH was then adjusted to 5.75 by careful addition of small portions (20 μL) of the aforementioned aqueous 2% Lactic Acid solution. 0.9% NaCl solution for injection was used to adjust to final volume of 25 mL. At that point dissolution of the compound of the invention was observed to be complete by visual examination and the solution was sterilized by passing the solution through a pre-sterilized syringe filter (e.g., Millipore filter-Durapore (PVDF), 0.22 μm), thus resulting into a 28 mg/mL solution.

From this stock 28 mg/mL solution, the diluted solutions presented in the following table were obtained in 10 mL volumetric flasks by following dilution steps with 0.9% NaCl for injection. In the table 4, the indicated dose corresponds to the assumption that the dosing volume for intravenous injection is 5 mL per kg.

TABLE 4

| | Compound Conc. | | | | |
|---|---|---|---|---|---|
| | 28 mg/mL | 24 mg/mL | 20 mg/mL | 16 mg/mL | 12 mg/mL |
| Dose | 140 mg/kg | 120 mg/kg | 100 mg/kg | 80 mg/kg | 60 mg/kg |
| Volume of 28 | NA | 8.571 mL | 7.143 mL | 5.714 mL | 4.886 mL |
| mg/mL stock | | | | | |
| solution | | | | | |
| Volume (adjusted | NA | Adjust to | Adjust to | Adjust to | Adjust to |
| to 10 mL) with | | 10 mL | 10 mL | 10 mL | 10 mL |
| 0.9% NaCl for | | | | | |
| injection | | | | | |

| | Target Conc. | | | | |
|---|---|---|---|---|---|
| | 8 mg/mL | 4 mg/mL | 2 mg/mL | 1 mg/mL | 0.5 mg/mL |
| Doses | 40 mg/kg | 20 mg/kg | 10 mg/kg | 5 mg/kg | 2.5 mg/kg |
| Volume of 28 | 2.857 mL | 1.429 mL | 0.714 mL | 0.357 mL | 0.179 mL |
| mg/mL stock | | | | | |
| solution | | | | | |
| Volume (adjusted | Adjust to | Adjust to | Adjust to | Adjust to | Adjust to |
| to 10 mL) with | 10 mL | 10 mL | 10 mL | 10 mL | 10 mL |
| 0.9% NaCl for | | | | | |
| injection | | | | | |

### EXAMPLE 9

### Hematotoxicity of N-{2-[2-(dimethylamino)ethyl]-1,3-dioxo-2,3-dihydro-1H-benzo[de]isoquinolin-5-yl}urea

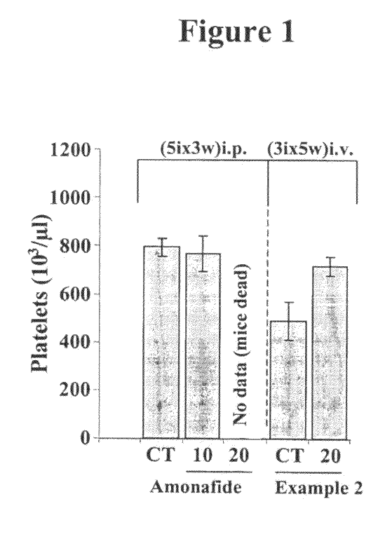

We have determined the compound-induced potential hematotoxicity of the compound produced according to example 2 or example 7 on platelets, red and white blood cells in comparison of the effect of amonafide on on platelets, red and white blood cells. The effect of amonafide was evaluated at 10 mg/kg and 20 mg/kg, by the intra-peritoneal administration to mice. The administration schedule was five times a week for three consecutive weeks. The effect of the compound produced according to example 2 or example 7 was evaluated at 20 mg/kg, by the intra-venous administration to mice. The administration schedule was three times a week (on Mondays, Wednesdays and Fridays) for five consecutive weeks. The animals were sacrificed 3 days after the last injection. There were 10 mice per group. FIG. 1 illustrates results of this assay for compound-induced hematotoxicity on platelets. FIG. 1 shows that the mice tolerated 15 chronic administrations of amonafide at a dose of 10 mg/kg, while all animals died before receiving the complete set of 15 administrations of amonafide at a dose of 20 mg/kg. In contrast, FIG. 1 shows that the mice tolerated 15 chronic administrations of the compound produced according to example 2 or example 7 at a dose of 20 mg/kg. Thus, unlike amonafide, the compound produced according to example 2 or example 7 was found not to provoke hematotoxicity at therapeutic doses in these experimental conditions.

### EXAMPLE 10

### Interaction of N-{2-[2-(dimethylamino)ethyl]-1,3-dioxo-2,3-dihydro-1H-benzo[de]isoquinolin-5-yl}urea with P-Glycoprotein

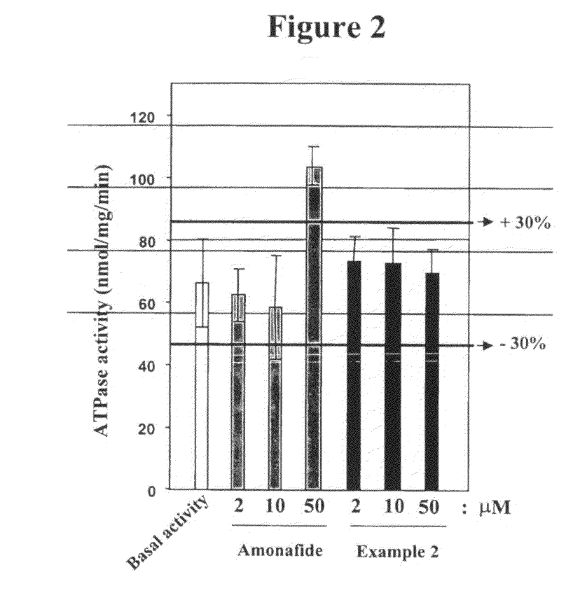

In order to test drug interaction with P-glucoprotein (herein-after referred as P-gp) we used an assay based on the study of modulation of ATPase activity from enriched P-gp membrane vesicle preparation (the following kit has been used: P-gp Drug interaction Assay Kit commercially available from SPI BIO France). P-gp ATPase activity was measured by a spectrophotometric method based on monitoring of ADP formation in the vesicle suspension medium. The basal ATPase activity was defined as the activity determined in the absence of any added drug. Modulation of basal activity was performed by adding amonafide or the compound produced according to example 2 or example 7 at different concentrations (2, 10, and 50 μM, respectively). The data shown in FIG. 2 indicate that, while amonafide when assayed at 50 μM significantly alter the ATPase activity, the compound of this invention does not affect ATPase activity, even.

### EXAMPLE 11

### Inducing Effect of N-{2-[2-(dimethylamino)ethyl]-1,3-dioxo-2,3-dihydro-1H-benzo[de]isoquinolin-5-yl}urea on Autophagy-Related Cell Death in Human Cancer Cells

A hallmark of topoisomerase II-targeting drugs is the induction of apoptosis; this is the consequence of an intracellular increase in the level of DNA damages by stabilization of the cleavable complex and/or a failure to achieve a complete chromosome segregation as a result of inhibition of topoisomerase II strand-passage activity. Amonafide is a topoisomerase II inhibitor and does induce apoptosis, a feature that we did not observe in human PC-3 (see Table 1) and DU-145 (ATCC Number: HTB-81) prostate cancer cells with the compound produced according to example 2 or example 7.

We used flow cytometry (according to the protocol found in Mijatovic et al. Neoplasia 2006) to determine the percentages of PC-3 and DU-145 positive cells for both Annexin V and propidium iodide and we observed that a maximum of 10% only of PC-3 or DU-145 cells underwent apoptotic processes following a treatment with 10 μM of the compound produced according to example 2 or example 7.

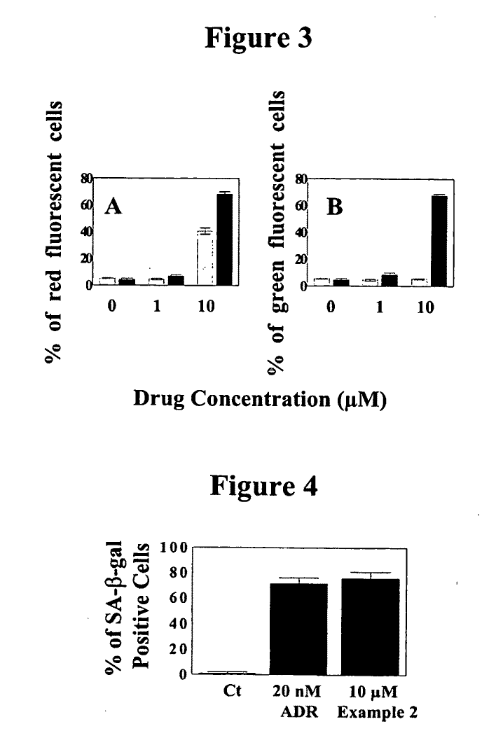

We observed pro-autophagic effects in PC-3 and in DU-145 cells treated with this compound. We quantified acidic vesicular organelles (revealed as red fluorescent staining) (according to the protocol found in Kanazawa et al. Cell Death Differ 2004), following acridine orange staining of PC-3 (gray bars) or DU-145 (black bars) cells after they have been treated with 0 (Control, untreated cells), 1 μM or 10 μM, and the consequent results are shown in FIG. 3A.

It is well known that lysosomes control cell death at several levels. In response to endogenous or exogenous stress (including chemotherapy), lysosomal membrane permeabilization (LMP) can occur, leading to the release of catabolic hydrolases that can mediate caspase-dependent apoptosis, caspase-independent apoptosis-like cell death or even necrosis following high levels of LMP. We thus quantified the “leakage” of acidic vesicular organelles (revealed as green fluorescent staining) (according to the protocol found in Nylandsted, J. et al. Heat Shock Protein 70 Promotes Cell Survival by Inhibiting Lysosomal Membrane Permeabilization, J Exp Med. (2004) 16; 200(4):425-35 and in Mijatovic et al., Neoplasia 2006) following treatment for 72 hours of PC-3 (gray bars) or DU-145 (black bars) cells after they have been treated with 0 (Control, untreated cells), 1 μM or 10 μM of the compound produced according to example 2 or example 7, and the consequent results are shown in FIG. 3B. We observed no LMP following treatment for 72 hours of PC-3 cells, while a marked drug-induced LMP process appeared in DU-145 cells when treated with 10 μM of a compound of this invention.

### EXAMPLE 12

### Inducing Effect of N-{2-[2-(dimethylamino)ethyl]-1,3-dioxo-2,3-dihydro-1H-benzo[de]isoquinolin-5-yl}urea on Senescence in DU-145 Human Prostate Cancer Cells

This feature that the compound produced according to example 2 or example 7 induced non-apoptotic cell deaths has been furthermore observed at the morphological level by means of cellular imaging in human PC-3 and DU-145 prostate to cancer cells treated with 10 μM of this compound for 6 days (cells seeded in Falcon flasks (25 cm²) and analysed for 6 days with quantitative video microscopy).

Senescence can be considered to be a type of “living cell death” because, although senescent cells maintain the integrity of their plasma membranes, they undergo permanent growth arrest and lose their clonogenicity. Senescence may act as a natural barrier to cancer progression.

Features typical of senescence have been induced by the compound of example 2 in human DU-145 prostate cancer cells. A senescent cell is known to typically show morphological changes, such as a flattened cytoplasm and increased granularity. The induction of senescence-associated β-galactosidase activity is a specific event occurring in cells undergoing senescence, a feature that we observed once more in the current study (according to the protocol found in Dimri et al., A biomarker that identifies senescent human cells in culture and in aging skin in vivo, Proc Natl Acad Sci USA. (1995) 92(20):9363-7), as evidenced in FIG. 4. Moderate doses (nM range) of doxorubicin (ADR) are known to induce senescence in wild-type human cancer cells. We therefore used doxorubicin as a positive control in our experiment. As shown in FIG. 4, 10 μM of the compound of this invention induced a similar percentage of senescence-associated β-galactosidase positive staining as 20 nM doxorubicin in DU-145 cells.

### EXAMPLE 13

### Identification of Genes Targeted by N-{2-[2-(dimethylamino)ethyl]-1,3-dioxo-2,3-dihydro-1H-benzo[de]isoquinolin-5-yl}urea

At the biochemical level, senescence is accompanied by changes in metabolism, a feature that we also observed in the current study when performing genomic analyses on PC-3 cells treated in vitro with the compound of example 2.

At the genetic level, we also observed alterations to chromatin structure and gene-expression patterns in PC-3 cells when treating them with this compound.

We performed a first experiment of evaluation of gene targets by means of Affymetrix whole genome microarray using human PC-3 cancer cells grown in vitro and treated with the compound of the invention (N-{2-[2-(dimethylamino)ethyl]-1,3-dioxo-2,3-dihydro-1H-benzo[de]isoquinolin-5-yl}urea) either one time at 1 μM or 10 μM, or 5 times a week (one time a day during five consecutive days) at 1 μM (Full genome analyses were performed at the VIB MicroArray Facility (UZ Gasthuisberg, Catholic University of Leuven, Belgium) using Affymetrix Human Genome U133 set Plus 2.0 (High Wycombe, United Kingdom). The most salient data that we obtained are reported in table 5 and indicate that this compound, when assayed as a single high dose in vitro (acute in vitro treatment), markedly modified the nuclear organization and biogenesis by increasing significantly the levels of expression, at least at the mRNA level, of various types of histones. The second set of genes targeted by this compound belongs to a category of genes labeled “amino acid metabolism” (Table 5).

In the process of identifying senescence-associated genes in prostate cancer cells, the prior art teaches significant suppression of the ets homologous factor (EHF) in cancer cells in a state of DNA damage-induced senescence, and has shown that EHF provides substantial drug resistance in PC-3 prostate cancer cells by inhibiting senescence and cell cycle arrest. Interestingly, we found EHF to also be a target for the compound of the invention.

The E2F family of transcription factors is known to play an important role in cell cycle progression. E2F-1, in heterodimeric complex with another protein DP-1, is normally inactive because it is bound to hypophosphorylated pRb. When cells progress from the G1 to the S phase, pRb becomes hyperphosphorylated and releases the bound E2F-1/DP-1 heterodimer, which subsequently activates the transcription of genes involved in DNA such as TS and DHFR. The loss of functional pRb can give rise to increased free E2F-1 levels, and subsequently increased levels of TS and DHFR. As revealed by the genomic Affymetrix approach, we found that the treatment of PC-3 cells with 1 μM of the compound of example 2 one time a day during five consecutive days decreased by two times the mRNA levels of E2F-1.

During the initial phases of senescence, Rb might control the nucleation of heterochromatin at specific sites throughout the genome, which then spreads by the action of histone methyltransferases and recruitment of HP1 proteins. We have found that the compound of example 2 markedly increased the levels of heterochromatin in PC-3 cells through an increase of histones H1, H2 and H3, at least at the mRNA levels, in PC-3 cells (Table 5). In contrast, this compound decreased by 2.6 times the levels of mRNA expression of H2AFY.

TABLE 5
Gene Targets Affected by N-{2-[2-(dimethylamino)ethyl]-1,3-dioxo-2,3-dihydro-1H-benzo[de]isoquinolin-5-yl}urea
in vitro Treatment of PC-3 Human Prostate Cancer Cells

| | Gene | List | | Population | | EASE | Bootsrap | | Expression Level: CT versus | | |
|---|---|---|---|---|---|---|---|---|---|---|---|
| System | Category | Hits | Total | Hits | Total | Score | Score | Genes | 10 μM | 5 × 1 μM | 1 μM |
| Biological | Nucleosome | 11 | 75 | 56 | 10401 | 5 × 10⁻¹² | 0.001 | H2AFY | 2.6 | NA | 1.7 |
| process | assembly | | | | | | | HIST1H3H | 0.3 | 0.6 | NA |
| | | | | | | | | HIST1H2BD | 0.3 | 0.3 | NA |
| | Chromatine | 11 | 75 | 85 | 10401 | 4 × 10⁻¹⁰ | 0.001 | HIST1H2AC | 0.4 | 0.2 | 1.0 |
| | assembly/ | | | | | | | HIST1H2BC | 0.4 | 0.7 | NA |
| | disassembly | | | | | | | HIST1H2BG | 0.4 | 0.5 | NA |
| | | | | | | | | HIST1H3D | 0.4 | 0.6 | 0.9 |
| | DNA packaging | 11 | 75 | 160 | 10401 | 2 × 10⁻⁷ | 0.001 | HIST2H2AA | 0.4 | 0.5 | NA |
| | | | | | | | | H2BFS | 0.5 | 0.5 | NA |
| | Nuclear | 11 | 75 | 180 | 10401 | 5 × 10⁻⁷ | 0.001 | HIST1H2BK | 0.5 | 0.5 | NA |
| | organization and | | | | | | | HIST2H2BE | 0.5 | 0.3 | NA |
| | biogenesis | | | | | | | | | | |
| KEGG | Amino acid | 8 | 15 | 236 | 1571 | 0.002 | 0.006 | ASNS | 3.6 | 0.9 | 1.3 |
| Pathway | metabolism | | | | | | | SARS | 2.6 | 0.9 | 1.0 |
| | | | | | | | | PHGDH | 2.4 | 1.0 | 1.4 |
| | | | | | | | | ALDH1A3 | 2.3 | 3.7 | 0.7 |
| | | | | | | | | CBS | 2.2 | 1.0 | 1.0 |
| | | | | | | | | BCAT1 | 2.1 | 0.8 | NA |
| | | | | | | | | CTH | 2.0 | 0.9 | 1.1 |
| | | | | | | | | SAT | 0.5 | 0.8 | 1.1 |

^{a} NA: Not available

1-3. (canceled)

4. A substituted naphthalimide derivative being N-{2-[2-(dimethylamino)ethyl]-1,3-dioxo-2,3-dihydro-1H-benzo[de]isoquinolin-5-yl}urea and/or a pharmaceutically acceptable salt thereof and/or a metabolite thereof.

5. A metabolite of the substituted naphthalimide derivative according to claim 4, wherein said metabolite is selected from the group consisting of: mono-N-oxides and di-N-oxides thereof.

6. A pharmaceutical composition comprising one or more pharmaceutically acceptable carriers and a therapeutically effective amount of the substituted naphthalimide derivative and/or a pharmaceutically acceptable salt thereof and/or a metabolite thereof of claim 4.

7. A pharmaceutical composition according to claim 6, further comprising an antineoplastic drug.

8. (canceled)

9. A method for making the substituted naphthalimide derivative according to claim 4, comprising the step of hydrolysing a compound having the structural formula (II) 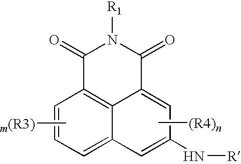
wherein:
each of the substituents R₃ and R₄ is independently selected from the group consisting of halogen C₁₋₄ alkyl, C₁₋₇ alkoxy, C₁₋₄ alkylthio, nitro, cyano, amino, protected amino, and halo C₁₋₄ alkyl;
n=0, and
m=0, and
R₁ is an alkylene radical having 2 carbon atoms and linked to a dimethylamino group and
R′ is C₁₋₄ alkoxyamidocarbonyl or C₁ haloalkylamidocarbonyl.

10. A method according to claim 9, wherein said compound having the structural formula (II) is the product of reacting amonafide with a C₁₋₄ alkoxycarbonyl isocyanate or a C₁ haloalkylcarbonyl isocyanate.

11. A method according to claim 10, wherein said reaction of amonafide with a C₁₋₄ alkoxycarbonyl isocyanate or a C₁ haloalkylcarbonyl isocyanate is performed in the presence of a solvent, said solvent being selected from the group consisting of ethers, ketones and halogenated hydrocarbons, and/or at a temperature below 0° C.

12. A method according to claim 10, wherein said reaction of amonafide with a C₁₋₄ alkoxycarbonyl isocyanate or a C₁ haloalkylcarbonyl isocyanate is performed in the presence of a molar excess of said C₁₋₄ alkoxycarbonyl isocyanate or C₁ haloalkylcarbonyl isocyanate, and wherein said reaction is quenched after completion by adding water to the reaction mixture.

13. A method according to claim 9, wherein hydrolysing a compound having the structural formula (II) is performed under basic conditions.

14. A method of treating a cell proliferative disorder comprising administering an effective amount of a substituted naphthalimide derivative, and/or a pharmaceutically acceptable salt thereof and/or a solvate thereof and/or a metabolite thereof, according to claim 4.

15. The method according to claim 14, wherein said cell proliferative disorder is selected from the group consisting of leukemia, lung cancer, colorectal cancer, central nervous system (CNS) cancer, melanoma, ovarian cancer, kidney cancer, prostate cancer, breast cancer, glioma, bladder cancer, bone cancer, sarcoma, head and neck cancer, liver cancer, testicular cancer, pancreatic cancer, stomach cancer, oesophageal cancer, bone marrow cancer, duodenum cancer, eye cancer (retinoblastoma) and lymphoma.

16. (canceled)

17. (canceled)

18. A method of treatment of a cell proliferative disorder according to claim 14, wherein said amount is from 0.01 mg to 20 mg per day per kg bodyweight.

19. A method of treatment of a cell proliferative disorder according to claim 14, further comprising administration of an effective amount of an antineoplastic drug.

20. A method of treatment of a cell proliferative disorder according to claim 14, wherein said administration is selected from intravenous administration, intramuscular administration, intraperitoneous administration, oral administration and rectal administration.

21. A method according to claim 11, wherein said reaction of amonafide with a C₁₋₄ alkoxycarbonyl isocyanate or a C₁ haloalkylcarbonyl isocyanate is performed in the presence of a molar excess of said C₁₋₄ alkoxycarbonyl isocyanate or C₁ haloalkylcarbonyl isocyanate, and wherein said reaction is quenched after completion by adding water to the reaction mixture.

22. A method according to claim 10, wherein hydrolysing a compound having the structural formula (II) is performed under basic conditions.

23. A method according to claim 11, wherein hydrolysing a compound having the structural formula (II) is performed under basic conditions.

24. A method according to claim 12, wherein hydrolysing a compound having the structural formula (II) is performed under basic conditions.

25. A method of treatment of a cell proliferative disorder according to claim 15, further comprising administration of an effective amount of an antineoplastic drug.
